(19) United States
(12) Patent Application Publication
(10) Pub. No.: US 2023/0272343 A1
(43) Pub. Date: Aug. 31, 2023

(54) ARTIFICIAL ANTIGEN PRESENTING CELL SYSTEM AND USES THEREOF

(71) Applicant: Celtec, Inc., New Taipei City (TW)

(72) Inventors: Che-Ming Jack Hu, Taipei (TW); Jung-Chen Lin, Taipei (TW); Chung-Yao Hsu, Taipei (TW)

(73) Assignee: Celtec, Inc., New Taipei City (TW)

(21) Appl. No.: 17/998,228

(22) PCT Filed: May 7, 2021
(86) PCT No.: PCT/CN2021/092173

§ 371 Date: Nov. 8, 2022

Related U.S. Application Data
(60) Provisional application No. 63/022,289, filed on May 8, 2020.

(51) Int. Cl. C12N 5/0784; A61K 9/50; A61K 9/16

(52) U.S. Cl. CPC C12N 5/0639; A61K 9/5068; A61K 9/1641; C12N 2533/30

(57) ABSTRACT

An artificial antigen presenting cell system comprising one or more gelated human dendritic cells and a controlled release system capable of releasing one or more cytokines. Also provided herein are methods for producing the gelated human dendritic cells and uses of the artificial antigen presenting cell system for activating immune cells.

### CROSS REFERENCE TO RELATED APPLICATION

This application claims benefit under 35 U.S.C. § 119(e) of the U.S. Provisional Application No. 63/022,289, filed May 8, 2020, the content of which is incorporated by reference herein in its entirety.

### BACKGROUND OF THE INVENTION

Direct modulation of T cells and their function via antigen presenting cells (APCs), including naturally occurring APCs or artificial APCs (aAPCs), is an attractive therapeutic strategy against diseases ranging from cancer, viral infection, and autoimmune diseases. Constantino et al., Transl Res 2016, 168, 74-95; and Palucka et al., Nat Rev Cancer 2012, 12 (4), 265-77. Recent clinical interest and advances in T cell-based adoptive cell therapies have led to further adoption of APCs for ex vivo T cell modulation, which allows for expansions of neoantigen-specific T cells, tumor infiltrating lymphocytes, virus-targeted T cells, as well as general T cell expansion for subsequent genetic modification. Constantino et al., 2016; and Fucikova et al., Front Immunol 2019, 10, 2393.

Development of artificial APCs was of interest due to maintenance, storage, and aseptic requirement considerations. Sunshine et al., Nanomedicine (Lond) 2013, 8 (7), 1173-89; Turtle et al., Cancer J 2010, 16 (4), 374-81; Dural et al., Cancer Immunol Immunother 2009, 58 (2), 209-20; Latouche et al., Nat Biotechnol 2000, 18 (4), 405-9; and Sasawatari et al., Immunol Cell Biol 2006, 84 (6), 512-21. However, it is still a challenge to construct aAPCs that can mimic naturally-occurring APCs such as live dendritic cells (DCs) to a great extent.

### SUMMARY OF THE INVENTION

The present disclosure is based, at least in part, on the development of an efficient artificial antigen presenting cell (aAPC) system that comprises (a) gelated human dendritic cells capable of presenting antigen peptides to T cells and (b) a controlled release system capable of secreting one or more cytokines. The human dendritic cells may be genetically modified. Alternatively, the human dendritic cells may be naturally-occurring. The aAPC system disclosed herein is capable of fully activate immune cells such as T cells via triggering three signaling pathways, including the primary signaling pathway triggered by the engagement of MHC/peptide complex with T cell receptors, the co-stimulatory signaling pathway triggered by interaction between a co-stimulatory receptor on T cells and a ligand thereof, and the signaling pathway triggered by binding of stimulatory cytokines to their receptors on the immune cells. Accordingly, one aspect of the present disclosure provides an artificial antigen presenting cell complex comprising: (a) one or more gelated human dendritic cells; and (b) a controlled release system associated with one or more cytokines. The one or more gelated human dendritic cells are attached to the controlled release system to form the artificial antigen presenting cell complex. In some embodiments, the artificial antigen presenting cell complex may further comprise an antigenic peptide displayed on the surface of the one or more gelated human dendritic cells.

In some embodiments, the controlled release system comprises a microparticle, which may encapsulate the one or more cytokines. Exemplary cytokines include, but are not limited to, IL-2, IL-3, IL-4, IL-6, IL-7, IL-9, IL-12, IL-15, IL21, IL-23, IL-10, TNF-α, IFN-α, IFN-β, IFN-γ, IFNλ, and/or granulocyte-macrophage colony-stimulating factor. In some embodiments, the microparticle may comprise one or more degradable polymers, for example, poly(lactic-co-glycolic acid), polylactic acid, polycaprolactone, polyurethane, polyacrylate, or a combination thereof.

In some embodiments, the antigenic peptide can be displayed by a major histocompatibility complex class I molecule on the gelated human dendritic cell. In some embodiments, the gelated human cells may comprise intracellular cross-linked polyethylene glycol diacrylate (PEG-DA).

In another aspect, the present disclosure features a method for producing a gelated human dendritic cell, the method comprising: (a) providing a population of human dendritic cells; (b) permeating a gelation buffer into the human dendritic cells, wherein the gelation buffer comprises a photo initiator, a polymer or a polymerizable monomer, and dimethyl sulfoxide; and (c) exposing the human dendritic cells permeated with the gelation buffer to a light to cause crosslinking the polymer, thereby forming the gelated human dendritic cells. In some instances, the method may further comprise (d) coupling the gelated human dendritic cells to a controlled release system, which is associated with one or more cytokines.

In some embodiments, the polymer in the gelation buffer can be an acrylic polymer, for example, poly(ethylene glycol) diacrylate (PEG-DA). In some examples, the acrylic polymer is PEG-DA, which may have an average molecular weight of about 250 to about 2,000 dalton. In other examples, the PEG-DA may have an average molecular weight of about 600 to about 800 dalton. In some embodiments, the gelation buffer may comprise a polymerizable monomer, for example, wh 2-hydroxyethyl methacrylate, (hydroxyethyl)methacrylate, acrylic acid, sodium acrylate, ethylene glycol dimethacrylate, or a combination thereof.

In some embodiments, the photo initiator is 2-hydroxy-4′-(2-hydroxyethoxy)-2-methylpropiophenone.

Also within the scope of the present disclosure is any of the artificial antigen presenting human dendritic cells produced by any of the methods disclosed herein.

In yet another aspect, the present disclosure provides a method of activating an immune cell, comprising: (i) providing any of the artificial antigen presenting cell complexes disclosed herein; (ii) priming the artificial antigen presenting cell complex with an antigenic peptide to display the peptide on the surface of the gelated human dendritic cell in the artificial antigen presenting cell complex; and (iii) contacting the artificial antigen presenting cell complex produced in step (ii) with a cell population comprising immune cells to activate the immune cells. Optionally, the method may further comprise administering the activated immune cells produced in step (iii) to a subject in need thereof.

In some embodiments, the immune cells comprise T cells. In some embodiments, the peptide is derived from a viral antigen, a bacterial antigen, a fungal antigen, a parasite antigen, a cancer antigen, or a self antigen associated with an autoimmune disease.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIGS. 1A-1D are diagrams showing preparation and characterization of gelated dendritic cells (G-DCs) via DMSO-mediated monomer and photoinitiator permeation. FIG. 1A is a schematic illustration showing preparation of gelated dendritic cells following direct permeation of hydrogel monomers and photoinitiators into the intracellular domain of cells in the presence of DMSO. A UV crosslinker is used for photo-activated crosslinking. FIG. 1B: PEG quantification of cells following incubation in 10 wt % PEG-DA gelation buffer shows that intracellular PEG-DA monomer concentration approaches saturation after 5 min of incubation.

FIG. 1C: Fluorescence and bright-field microscopic observations of gelated dendritic cells prepared with fluorescein-DA show that gelated dendritic cells retain structural integrity upon suspension in PBS and in water. FIG. 1D: The stability of G-DCs is assessed following 21 days of suspension in PBS.

FIGS. 2A-2F are diagrams showing that G-DC antigens presentation can be modulated by peptide pulsing post intracellular hydrogelation. FIG. 2A: A schematic illustration showing that peptide antigens can be added to JAWSII cells after intracellular hydrogelation. FIGS. 2B-2C: JAWSII cells were treated with LPS and then gelated by a photoactivated hydrogel system. In the non-activated G-DC group, control JAWSII cells without peptide and LPS activation were prepared for gelation. In the activated G-DC group, SIINFEKL peptides were added during the LPS activation of JAWSII before intracellular hydrogelation. In LPS-treated G-DC group, JAWSII were activated in the absence of SIINFEKL peptides and SIINFEKL peptides were added to the LPS-treated G-DC add OT-I peptide group post intracellular hydrogelation. The H-2K^{b}/SIINFEKL complexes on the surfaces of all groups were observed by flow cytometry. Control groups, including non-activated, activated and LPS-treated cells, were also gelated for surface marker comparison. The histogram (FIG. 2B) and bar graph (FIG. 2C) show the % of H-2K^{b}/SIINFEKL complexes on the harvested CD8⁺ cell population. Unstained T-cell groups (shown as solid filled in gray histogram) were used as a negative control flow cytometry. Error bars represent mean±SEM, n=3. (FIGS. 2D-2F) Co-culture of peptides-exchanged G-DCs and their control groups with CFSE-stained OT-I-specific CD8+ T cells showed T-cell expansion by the peptides-exchanged and activated G-DCs but not by the non-activated and LPS-treated G-DCs. Histogram shows the CFSE proliferation profile and the CFSE dilution peaks show the extent of cell division (FIG. 2D). The bar graph shows the percentage of cells in different generations of division (FIG. 2E). Cell numbers and relative fold-change following co-culturing with different G-DCs are shown in (FIG. 2F). Each culture condition contained a fixed number of DCs at 8×10⁴ per well with a T-cell/G-DC ratio of 3:1. The arrangement of the bars in the bar graphs, from left to right, are non-activated, LPS-treated and OTI-pulsed, LPS-treated only, and LPS-treated and OTI-pulsed post-gelation.

FIGS. 3A-3F are diagrams showing that CD80 are presented on the surface of peptide-exchanged G-DCs after G-DCs stored by both freezing and lyophilization. Gelated JAWSII cells prepared with 10 wt % PEG-DA were frozen at −20° C. (FIGS. 3C and 3D), or lyophilized (FIGS. 3E and 3F) in 10% sucrose. After 72 hr of storage, the G-DCs were observed upon thawing or resuspension in water. Expression of MHC-class I/SIINFEKL complexes on cell surface were detected by flow cytometry. The histograms (FIGS. 3A, 3C and 3E) and bar graphs (FIGS. 3B, 3D, and 3F) show the % of max in the harvested cell population. FIGS. 3A and 3B are the control, non-frozen or non-lyophilized cells. Similar patterns were observed in the LPS-treated and OTI-pulsed, LPS-treated and LPS-treated and OTI pulse post-gelation G-DCs. The arrangement of the bars in the bar graphs, from left to right, are non-activated, LPS-treated and OTI-pulsed, LPS-treated only, and LPS-treated and OTI-pulsed post-gelation.

FIGS. 4A-4G are diagrams showing that G-DC functionality is retained following storage upon freezing or lyophilization. FIG. 4A: A schematic illustration showing storage by freezing or by lyophilization for the gelated JAWSII. In the non-activated G-DC group, control JAWSII cells without peptide and LPS activation were prepared for gelation. In the activated G-DC group, SIINFEKL peptides were added during the LPS activation of JAWSII before intracellular hydrogelation. In LPS-treated G-DC group, JAWSII were activated in the absence of SIINFEKL peptides and SIINFEKL peptides were added to the LPS-treated G-DC post intracellular hydrogelation. Bright-field microscopy of control cells and different G-DCs suspended in PBS. The sphericity of G-DCs following different storage and resuspension was quantified using ImageJ. FIGS. 4B-4D: The different G-DCs were frozen at −80 C and thawed for T cell expansion. Co-culture of all groups with CFSE-stained OT-I-specific CD8+ T cells showed T-cell expansion by the activated G-DCs and post-pulsing group but not by the non-activated and LPS-treated G-DCs. Histogram showed that CFSE proliferation profile and the CFSE dilution peaks showed divided times. The bar graph showed the percent in generation and cell numbers in all groups. Each culture condition contained a fixed number of DCs at 8×10⁴ per well with a T-cell/G-DC ratio of 3:1. Stained control T cells are plotted in black as a reference. FIGS. 4E-4G: The different G-DCs were lyophilized and then reconstituted in medium for T cell expansion. Error bars represent mean±SEM, n=3. The arrangement of the bars in the bar graphs, from left to right, are non-activated, LPS-treated and OTI-pulsed, LPS-treated only, and LPS-treated and OTI-pulsed post-gelation.

FIGS. 5A-5D include diagrams showing that MHC class-I/SIINFEKL complexes are presented on the surface of peptide-exchanged G-DCs after G-DCs stored by both freezing and lyophilization. Gelated JAWSII cells prepared with 10 wt % PEG-DA were frozen at −20° C., or lyophilized in 10% sucrose. After 72 hr of storage, the G-DCs were observed upon thawing or resuspension in water. Expression of MHC-class I/SIINFEKL complexes on cell surface were detected by flow cytometry. The histogram (FIGS. 5A and 5C) and bar graph (FIGS. 5B and 5D) show the % of max in the harvested cell population. Similar patterns were observed between the LPS-treated and OTI-pulsed and LPS-treated and OTI pulse post-gelation G-DCs. The arrangement of the bars in the bar graphs, from left to right, are non-activated, LPS-treated and OTI-pulsed, LPS-treated only, and LPS-treated and OTI-pulsed post-gelation.

FIG. 6 include photos showing that G-DCs can be successfully stored by both freezing and lyophilization. Gelated JAWSII cells infused with 10 wt % PEG-DA were frozen in 10% sucrose at −20° C., or lyophilized in 10% sucrose. After 72 hr of storage, the G-DCs were observed upon thawing or resuspension in H₂O. No discernable change in morphology was observed with the G-DCs under the different storage conditions. Scale bars=100 μm.

FIGS. 7A-7E include diagrams showing preparation and characterization of G-hDCs following storage upon freezing or lyophilization. FIG. 7A: A schematic illustration showing preparation of G-hDCs following photo-activated crosslinking. FIG. 7B: G-hDCs were imaged using a microscope following storage upon freezing or lyophilization. FIG. 7C: Fluorescence and bright-field microscopic observations of G-hDCs prepared with fluorescein-DA show that G-hDCs retain structural integrity upon suspension in PBS and in water. FIGS. 7D-7E: hDCs were gelated by a photoactivated hydrogel system. The expression of surface markers on cell surface were detected by flow cytometry. The histogram and bar graph show the % of human MHC class I and MHC class II (FIG. 7D) as well as CD80 and CD86 (FIG. 7E) complexes on the harvested CD8+ cell population. Unstained T-cell groups (shown as solid filled in gray histogram) were used as a negative control flow cytometry. Error bars represent mean±SEM, n=3.

FIGS. 8A-8E include diagrams showing that G-DCs are adaptable for the preparation of cytokine-releasing, antigen presenting spheroids. FIG. 8A: Schematics showing the construction of an artificial antigen presenting cells by integrating G-DCs with cytokine-loaded microparticles (GC-MPs). FIG. 8B: A representative GC-MP was observed under confocal microscopy. PLGA-based MPs were synthesized by mixing PLGA and sulfo-cy5 using a double emulsion process. MPs were imaged using a fluorescence microscope. After coating with poly-L-lysine, MPs were mixed with GCs and then stained with a carbocyanine membrane dye, DiO. GC-MPs were imaged using a confocal microscope. FIG. 8C: The size distribution of GC-MP was determined through ImageJ analysis of microscopy images. FIG. 8D: The GC-MPs are highly stable and retain structural integrity upon 30-day of observation. FIG. 8E: In vitro release profiles of GC-MPs was performed at 4° C. and 37° C.

FIG. 9 is a chart showing release kinetics by microparticles of different compositions at 37 and 4 C.

FIGS. 10A-10E include diagrams showing expansion of antigen-specific T cells by GC-MPs for anti-tumor therapy. JAWSII cells were treated with LPS and OTI peptide pulsing for activation and then gelated by a photoactivated hydrogel system. FIGS. 10A-10C: Co-culture of CFSE-stained OT-I-specific CD8+ T cells with medium only, IL2-loaded microparticle (MP-IL-2; no GC coating), GC-MPs (no IL-2), or GC-MP-IL-2 showed T-cell expansion by the GC-MPs and GC-MP-IL-2. FIG. 10A: Histogram shows the CFSE proliferation profile and the CFSE dilution peaks represent the level of division. FIG. 10B: The bar graph shows the percent of cells in different generations of cells. FIG. 10C: Relative fold-change of CD8+ T cells in all groups following 3 and 6 days of co-culturing with the different aAPC systems. FIG. 10D: A schematic illustration showing T cell expansion and administration scheme for EG7-OVA tumor treatment. FIG. 10E: E.G7-OVA cells (5×10⁵ cells) were subcutaneously implanted under the skin in the dorsal flank regions and expanded antigen-specific CD8⁺ T cells were administered by i.v. on day 0. Tumor size was monitored and calculated as (W²×L)/2, where W is width and L is length. Each culture condition contained a fixed number of DCs at 8×10⁴ per well with a T-cell/G-DC ratio of 3:1. Error bars represent mean±SEM, n=3.

### DETAILED DESCRIPTION OF THE INVENTION

As opposed to the conventional synthetic approaches for making artificial antigen presenting cells (aAPCs), the present disclosure relates to approaches for converting live human dendritic cells to aAPCs via intracellular gelation. The gelated human dendritic cells can be associated with a controlled release system capable of secreting cytokines to form an artificial antigen presenting cell complex, which is capable of fully activate immune cells as disclosed herein. The method for making gelated cells has successfully transformed the JAWSII murine DC cell line and human primary DCs into robust aAPCs capable of being stored via freezing or lyophilization. Using JAWSII-derived aAPCs, it has been demonstrated the modularity of the construct via peptide antigen exchange and preparation of gelated cellular spheroids capable of sustained cytokine release through microparticle coupling. The DC-derived aAPCs successfully triggered expansion of antigen-specific T cell and improved the anticancer efficacy of adoptive T cell therapy in mice.

Accordingly, disclosed herein are artificial antigen presenting cell systems comprising gelated human dendritic cells, which may be converted from live human dendritic cells, and optionally a controlled release system for secreting cytokines; methods for producing gelated human dendritic cells from live human dendritic cells; and methods for activating immune cells such as T cells using the aAPC systems disclosed herein.

### I. Artificial Antigen Presenting Cell Complex

In some aspects, the present disclosure provides an artificial antigen presenting cell (aAPC) complex comprising one or more gelated human dendritic cells derived from live human dendritic cells and a controlled release system encompassing one or more cytokines. The aAPC complex disclosed is capable of fully activate immune cells.

(i) Gelated Human Dendritic Cells

The gelated human dendritic cells disclosed herein can be converted from live human dendritic cells via intracellular gelation. As used herein, a gelated human dendritic cell refers to a human dendritic cell comprising intracellular cross-linked polymers (i.e., gelation), which are non-native to naturally-occurring human dendritic cells. The gelated human dendritic cells may be converted from human dendritic cells obtained from a human donor. Alternatively, the gelated human dendritic cells may be converted from a human dendritic cell line cultured in vitro.

Preferably, the cross-linked polymers in the gelated human dendritic cells are biocompatible. Any biocompatible polymers known in the art can be used for making the gelated human dendritic cells. One example is polyethylene glycol (PEG), which may comprise a functional group, for example, an acrylic functional group. Another example is N(2-hydropropyl)methacrylamide (HPMA).

In some embodiments, the gelated human dendritic cells comprise an antigenic peptide complexed with a MHC molecule expressed on the surface of the dendritic cells. In some examples, the antigenic peptide is displayed on the surface of the gelated dendritic cells by an MHC class I molecule. In other examples, the antigenic peptide is displayed on the surface of the gelated dendritic cells by an MHC class II molecule. The antigenic peptide may be a fragment of a viral antigen, a bacterial antigen, a fungal antigen, a parasite antigen, a cancer antigen, or a self antigen associated with autoimmune disease. In some instances, the antigenic peptide can be a MHC Class-I restricted peptide. For example, the antigenic peptide may be restricted to HLA-A (e.g., A*0201 or A*2402), HLA-B, or HLA-C. In other instances, the antigenic peptide may be a MHC Class-II restricted peptide. For example, the antigenic peptide may be restricted to HLA-DPA1, HLA-DPB1, HLA-DQA1, HLA-DQB1, HLA-DRA, or HLA-DRB1. Gelated human dendritic cells expressing one or more specific MHC Class-I molecules and/or MHC Class II molecules can be obtained from human donors having the required MHC molecules.

(ii) Controlled Release System for Cytokine Secretion The artificial antigen presenting cell complex disclosed herein may further comprise a controlled release system associated with one or more of the gelated human dendritic cells disclosed herein. The controlled release system is associated with one or more cytokines, for example, encapsulating one or more of the cytokines. A controlled release system is a delivery system that is capable of releasing the associated cytokines at a controlled rate such that the one or more cytokines can be releases over an extended period of time. In some embodiments, the controlled release system comprise one or more biocompatible polymers (e.g., natural or synthetic) that are judiciously combined with the one or more cytokines in such a way that the one or more cytokines can be released in a predesigned manner. The release of the cytokines may be constant over a long period. Alternatively, the release of the cytokines may be cyclic over a long period, or triggered by the environment or other external events. The controlled release system may comprise one or more biocompatible materials to which the cytokines can be associated via covalent or non-covalent interactions (e.g., electrostatic interaction or hydrogen bonding). The biocompatible materials may be polymers, for example, hydrophilic polymers that form hydrogels. A hydrogel is a network of polymer chains that are hydrophilic. In a hydrogel network, the hydrophilic polymers can form a three-dimensional structure by cross-linking of the polymers.

In some embodiments, the controlled release system disclosed herein comprises microparticles or nanoparticles associated with the one or more cytokines. In some instances, the microparticles may encapsulate the cytokines. In other instances, the cytokines may be attached to the microparticles. The microparticles may be made by one or more suitable polymers, including polymers commonly used in controlled release drug delivery systems. In some examples, the polymer for making the microparticles is biodegradable. Examples include, but are not limited to, poly(lactic-co-glycolic acid), polylactic acid, polycaprolactone, polyurethane, polyacrylate, or a combination thereof.

The microparticles may be of a suitable size allowing for one or more of the gelated human dendritic cells to attach to it. For example, the microparticles may have an average size of about 1 to about 1,000 micro, for example, about 10-100, about 100-200, about 200-400, about 400-600, about 600-800, or about 800-1,000 microns. In some examples, the average size of the microparticles disclosed herein may be about 50-100 microcons, e.g., about 75 micron.

In other embodiments, the controlled release system disclosed herein may comprise liposomes or a planar substrate associated with the cytokines.

The cytokines contained in the controlled release system may comprise those that can activate immune cells such as T cells. Examples include, but are not limited to, IL-2, IL-3, IL-4, IL-6, IL-7, IL-9, IL-12, IL-15, IL21, IL-23, IL-10, TNF-α, IFN-α, IFN-β, IFN-γ, IFN-λ, granulocyte-macrophage colony-stimulating factor (GM-CSF), or a combination thereof.

Any of the controlled release systems disclosed here, for example, the microparticles, may be prepared by conventional methods. See, e.g., Example 2 below. The controlled release system can then be coupled with any of the gelated human dendritic cells either directly or via a linker, for example, a poly-lysine molecule.

### II. Methods for Producing Gelated Human Dendritic Cells

Also provided herein is a method for producing gelated human dendritic cells, such as those disclosed herein. To make gelated cells having intracellular crosslinking of biocompatible polymers, the polymers needs to be delivered into cells for in-situ polymerization without disrupting the cell membrane structure such that the resultant gelated cells would maintain cell morphology and certain functions, e.g., antigen presenting. Traditionally, the freeze-thaw approach was used to infuse cells with the polymers. See, e.g., WO2018/026644. This approach has limitations as it is not suitable for fragile cells and could impose scalability issued. Differently, the method disclosed herein involves the use of a DMSO-mediated polymer infusion approach. Surprisingly, it was shown herein that in the presence of DMSO, low-molecular-weight poly(ethylene glycol) diacrylate (PEG-DA) successfully permeated through the plasma membrane of live human dendritic cells with high efficiency. The resultant gelated dendritic cells showed antigen-presenting activity, demonstrating that the DMSO-mediated infusion approach maintained integrity of the membrane structure of the gelated cells.

Accordingly, provided herein is a method for producing a gelated human dendritic cell, the method comprising: (a) providing a population of human dendritic cells; (b) permeating a gelation buffer into the human dendritic cells, wherein the gelation buffer comprises a photo initiator, a polymer, and dimethyl sulfoxide; and (c) exposing the human dendritic cells permeated with the gelation buffer to a light to cause crosslinking the polymer or a polymerizable monomer, thereby forming the gelated human dendritic cells. In some embodiments, the method may further comprise coupling the gelated human dendritic cells to a controlled release system, which is associated with one or more cytokines.

The population of human dendritic cells can be derived from humor donors. For example, the human dendritic cells may be isolated or enriched from human blood cells or human bone marrow cells. Alternatively, the population of human dendritic cells can be in vitro cultured DC cell lines.

The gelation buffer may comprise a photo initiator, a polymer or a polymerizable monomer, and dimethyl sulfoxide (DMSO). In some embodiments, the gelation buffer comprises a polymer comprising a crosslinking moiety, which can react with each other to lead to crosslinking of the polymers upon activation. Any biocompatible polymer that can be crosslinked in the presence of a reactive species such as a free radical to form gel may be used for making the gelated human dendritic cells disclosed herein. For example, the polymer may form crosslinking triggered by heat, photo, certain pH conditions, or a chemical. In some instances, the polymer comprises a crosslinking moiety, for example, an acrylic polymer. In some examples, the acrylic polymer may comprise a diacrylate moiety. In specific examples, the acrylic polymer is poly(ethylene glycol) diacrylate (PEG-DA), for example, PEG-DA having an average molecular weight of about 250-2,000 dalton. In some examples, the PEG-DA used herein may have an average molecular weight of about 600 to about 800 dalton (e.g., about 700 dalton). Other exemplary polymers for use in making the gelated human dendritic cells include polyacrylamide derivatives such as PEG-PLGA-PEG triblock copolymers, hydroxy ethyl methacrylate-methyl methacrylate (HEMA-MMA), polyacrylonitrile-poly vinyl chloride (PAN-PVC), poly(N-isopropyl acrylamide) (polyNIPAM), poly(N-vinylcaprolactam), cellulose derivatives, ethylene oxide-propylene, or Matrigel.

In other embodiments, the gelation buffer may comprise a polymerizable monomer, which is any monomer that is capable of forming a polymer or a hydrogel upon activation by, e.g., in the presence of a reactive species such as a free radical. Examples of polymerizable monomers include, but are not limited to, an acrylate or diacrylate compound. Examples include, but are not limited to, 2-hydroxyethyl methacrylate, (hydroxyethyl)methacrylate, acrylic acid, sodium acrylate, and ethylene glycol dimethacrylate. Upon activation, such polymerizable monomers can form the corresponding polymer, for example, poly(2-hydroxyethylmethacrylate, poly(acrylic acid), or poly(ethylene glycol dimethacrylate).

A photo initiator is a molecule that creates reactive species, for example, free radicals, cations, or anions when exposed to an energy source (e.g., light such as visible or UV, heat, etc). Any phot initiator may be used in the method disclosed herein. Examples of photoinitiators include, but are not limited to, cationic photoinitiators (e.g., onium salts, organometallic, and pyridinium salts), and free radical phtoinitiators (e.g., benzophenone, xanthones, quinones, benzoin ethers, acetophenones, benzoyl oximes, and acylphosphines). In one specific example, the photo initiator can be 2-hydroxy-4′-(2-hydroxyethoxy)-2-methylpropiophenone. Other photo initiators can be found, e.g., at sigmaaldrich.com/content/dam/sigma-aldrich/docs/Aldrich/General_Information/photoinitiators.pdf, the relevant disclosures of which are incorporated by reference for the purpose and subject matter referenced herein.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

When the photo initiator, the polymer or the polymerizable monomer enters into the human dendritic cells, the cells can be exposed to an energy source, under which the photo initiator releases reactive species such as free radicals to trigger crosslinking of the polymers, thereby producing the gelated human dendritic cells. When polymers comprising a crosslinking moiety is used, the crosslinking can occur via the crosslinking moieties. Alternatively, when a crosslinking agent is used, the crosslinking agent may mediate crosslinking of the polymers. The energy source can be a light, such as a UV light or a visible light.

Any of the gelated human dendritic cells produced by the methods disclosed herein are also within the scope of the present disclosure. Such gelated human dendritic cells may be incubated with any of the controlled release systems disclosed under suitable conditions for a suitable period to allow for complexing the gelated cells with the controlled release system, thereby producing the artificial antigen presenting cell complex disclosed herein. This coupling process can be carried out in the presence of a poly-lysine molecule.

### III. Use of Artificial Antigen Presenting Cell Complex for Immune Cell Activation

Any of the artificial antigen presenting cell complexes disclosed herein may be used to activate immune cells such as T cells. Thus, also provided herein are methods of activating an immune cell, comprising: (i) providing any of the artificial antigen presenting cell complex disclosed herein; (ii) priming the artificial antigen presenting cell complex with an antigenic peptide to display the peptide on the surface of the gelated human dendritic antigen presenting cell in the artificial antigen presenting cell complex; and (iii) contacting the artificial antigen presenting cell complex produced in step (ii) with a cell population comprising immune cells to activate the immune cells.

Selection of the peptide to prime the aAPC complex would depend on the specificity of the immune cells such as T cells to be activated. For example, if anti-tumor T cells are to be activated, an antigenic peptide derived from a tumor antigen can be used to prime the aAPCs. When a target peptide is determined, a suitable aAPC can be used to present the target peptide to T cells for activation. The suitable aAPC complex would comprise gelated human dendritic cells having a suitable MHC Class-I or MHC Class-II molecule capable of forming a MHC/peptide complex for antigen presenting. Selecting a suitable aAPC complex based on the target peptide and the immune cells for activation would be within the knowledge of those skilled in the art.

In some embodiments, an antigenic peptide derived from a tumor antigen is used to prime the aAPC complex for activating anti-tumor T cells. The resultant activated anti-tumor T cells can then be used for treating tumor carrying the tumor antigen.

In some embodiments, an antigenic peptide derived from a pathogen antigen (e.g., a viral antigen, a bacterial antigen, a fungal antigen, or a parasite antigen) may be used to prime the aAPC complex to activate T cells specific to the pathogen. The resultant activated T cells can be used for treating infection caused by the pathogen.

In some embodiments, an antigenic peptide derived from a self-antigen associated with an autoimmune disease may be used to prime the aAPC complex to activate T cells, for example, regulatory T cells (e.g., FoxP3⁺ regulatory T cells), which can suppress stimulatory autoreactive helper T cells and cytotoxic T cells. The aAPC complex can be used to activate such regulatory T cells via surface displayed MHC-II/peptide complex and optionally release immune suppressive cytokines, for example, IL10 and TGF-beta).

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds.(1985»; Transcription and Translation (B. D. Hames & S. J. Higgins, eds. (1984»; Animal Cell Culture (R. I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1. Preparation of Gelated Dendritic Cells Via Intracellular Hydrogelation and Characterization of the Gelated Dendritic Cells

The development of immunotherapies and adoptive cell therapies has prompted increasing interest in biomaterials development mimicking natural antigen-presenting cells for T cell activation and expansion. In contrast to conventional bottom-up approaches, which are aimed at layering synthetic substrates with T cell activation cues, this study demonstrates a facile hydrogelation technique to directly transform live dendritic cells (DCs) into artificial antigen presenting cells (aAPCs). Through DMSO-mediated intracellular hydrogel monomer permeation and UV-activated radical polymerization, intracellular hydrogelation is rapidly occurred in the DCs, yielding highly robust and modular cell-gel hybrid constructs amenable to peptide antigen exchange, storable by freezing and lyophilization.

### Materials and Methods

(i) Intracellular Gelation

Gelation buffers were prepared with 20 μL of 1.5 g/mL of 2-hydroxy-4′-(2-hydroxyethoxy)-2-methylpropiophenone (Irgacure D-2959; Sigma-Aldrich) dissolved in dimethyl sulfoxide (“DMSO”) and mixed with 200 μL poly(ethylene glycol)-diacrylate (“PEG-DA”), which was commercially available from Sigma-Aldrich. The PEG-DA has an average molecular weight (Mn) of 700 Da. 5×10⁶ JAWSII cells were collected and suspended in 1 mL phenol-red free Dulbecco's Modified Eagle Medium (“DMEM”) containing 1X protease inhibitor. The DMEM was commercially available from ThermoFisher Scientific under number CA21063-029. To the cell suspension, the gelation buffer was added at a 1:10 volume ratio to reach a 10 wt. % PEG-DA concentration in the cell suspension. Following 5 minutes of incubation at room temperature, the cells were pelleted and re-suspend in 500 μl pheno-red free DMEM without gelation buffer and subjected to 365 nm blue-light bombardment for 5 min in an UV oven (UVP Crosslinker, Analytik Jena, US). The resulting gelated cells (“GCs”) were washed with phosphate-buffered saline (“PBS”) prior to further experimentation.

For fluorescent labeling of the hydrogel, 100 μL of 18 mg/mL of fluorescein O,O′-diacrylate, which was available from Sigma-Aldrich, was dissolved in DMSO and mixed with PEG-DA and then Irgacure D-2959 as above. The fluorescent gelation buffers were mixed with phenol-red free DMEM and collected supernatant for cells gelation.

(ii) Quantification of Intracellular PEG-DA

Quantification of intracellular PEG-DA concentrations was performed using an iodine-based quantification method. In order to generate G-DCs, 4×105 JAWSII cells were suspended with 1 mL phenol-red free DMEM containing 10 wt. % gelation buffer at indicated time. Following G-DCs washed with PBS, intracellular PEG-DA were released from G-DCs using 1 mL of distill water treatment and undergone freeze-thaw process. The cell debris were spun down via centrifugation at 3000×g for 5 min. The supernatants were collected and mixed with BaCl2 and iodine solutions in an 8:2:1 ratio for 15 min color development. PEG-DA concentrations in the samples were determined by measuring the light absorbance at 535 nm. The standard curve was prepared with serially diluted PEG-DA. The measured PEG-DA content was then divided by the total volume of JAWSII cells to determine the intracellular PEG-DA concentration.

(iii) T-Cell Isolation and Fluorescence Labeling

OT-I cells (CD8+ T cells specific for the OVA257-264 peptide in the H2-K^{b} context) were isolated from OT-I transgenic mice. After mice were sacrificed, their spleens were removed and placed into RPMI1640 complete medium with 10% FBS. In order to harvest single splenocytes, the spleens were ground and strained with 5 ml syringe against a sterile 40 μm nylon cell strainer (BD Biosciences Falcon, #352340). Splenocytes were incubated with BD Pharm Lyse lysing buffer (BD Biosciences, #555899) for 3 min for RBCs depletion. OT-I cells were subsequently isolated from the splenocytes by Mouse CD8a+ T Cell Isolation Kit (BD Biosciences, #19853 A). For the labeling of OT-I cells. OT-I T cells were stained with carboxyfluorescein diacetate succinimidyl ester (CFSE) by incubating the cells with PBS containing 5 μM of CFSE (Sigma-Aldrich, #21888) at 37° C. for 5 min. The cells were washed three times with complete medium. CFSE-labeled OT-I cells were harvested for following experimental studies.

### Results

(i) Facile Intracellular Hydrogelation Via DMSO-Mediated Hydrogel Infusion

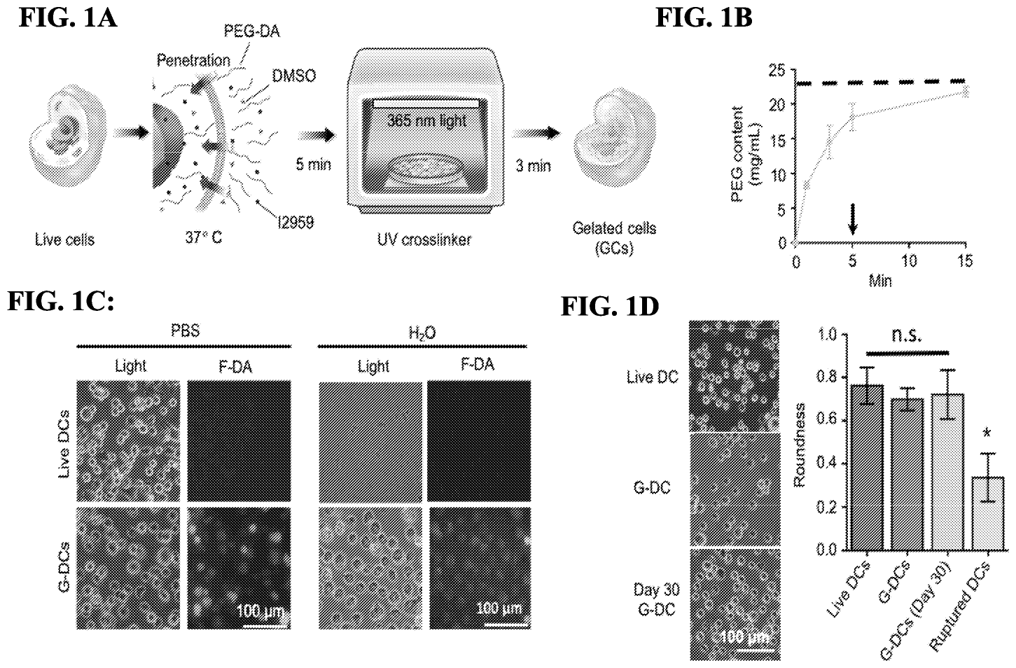

Gelated cells (GCs) were prepared by direct permeation of gelation buffer contained DMSO-mediated 2-hydroxyl-4′-(2-hydroxyethoxy)-2-methylpropiophenone photoinitiator (12959) and poly(ethylene glycol) diacrylate monomer (PEG-DA; Mn 700) following transient UV exposure for hydrogel crosslink on ice plate (FIG. 1A). To assess the feasibility of intracellular gelation for cellular fixation, we prepared gelated dendritic cells (G-DCs) with 10 wt % using JAWSII murine DCs. Cells were nearly fully infiltrated to the theoretical total intracellular volume with PEG-DA after only 5 min of incubation (FIG. 1B). To visualize the hydrogels inside the cell, G-GCs were prepared by gelation buffer which contained fluorescein-diacrylate (F-DA) that covalently imbue to hydrogels with green fluorescence. Following membrane staining with F-DA, GCs had hydrogel components interior (FIG. 1C). Compared to live DCs, there is no significantly morphological change in G-DCs under light microscope (FIG. 1C). Further, G-DCs were stable in water under low osmotic pressure but live DCs turned ruptured at once, showing that the structural integrity in G-GCs can be preserved by the hydrogel network formation inside (FIG. 1C). Moreover, G-DCs have retained structural integrity and morphology after 30 days of suspension in PBS but live DCs were disintegrated, indicating that G-DCs are more stable than live DC (FIG. 1D).

To sum up, these results show that the gelation approach disclosure herein is an efficient way to produce G-GCs that are stable under harsh condition and retain the cell membrane integrity for intended bioactivities such as antigen presentation.

(ii) G-DC Cells Successfully Presented Antigens on the Surface Through Peptide Replacement Post Intracellular Hydrogelation

Live dendritic cells were successfully transformed into gelated dendritic cells (G-DCs) following the procedures disclosed here (see, e.g., FIG. 1). Antigen presentation by the G-DCs via formation an MHC class I-peptide complex post intracellular hydrogelation was investigated. G-DCs were treated with medium, LPS only, and LPS-OTI peptides following hydrogelation. After the treatment by LPS, DCs were exposed to the OTI peptides post intracellular hydrogelation. This group is designated as LPS-treated and OTI pulse post-gelation group.

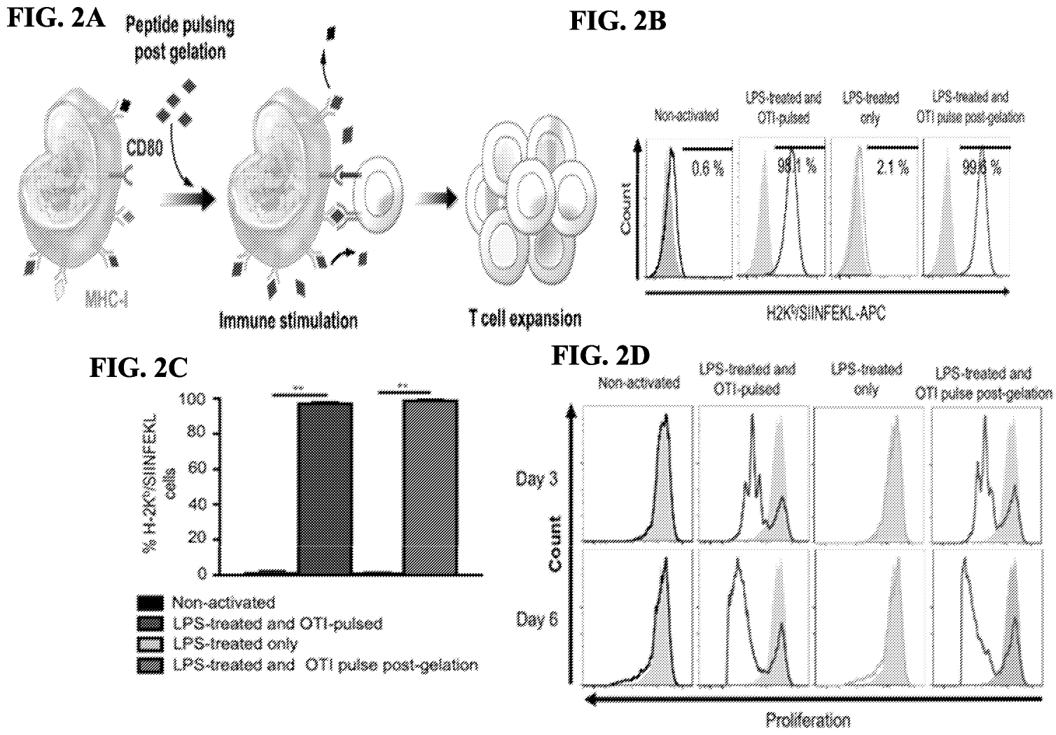

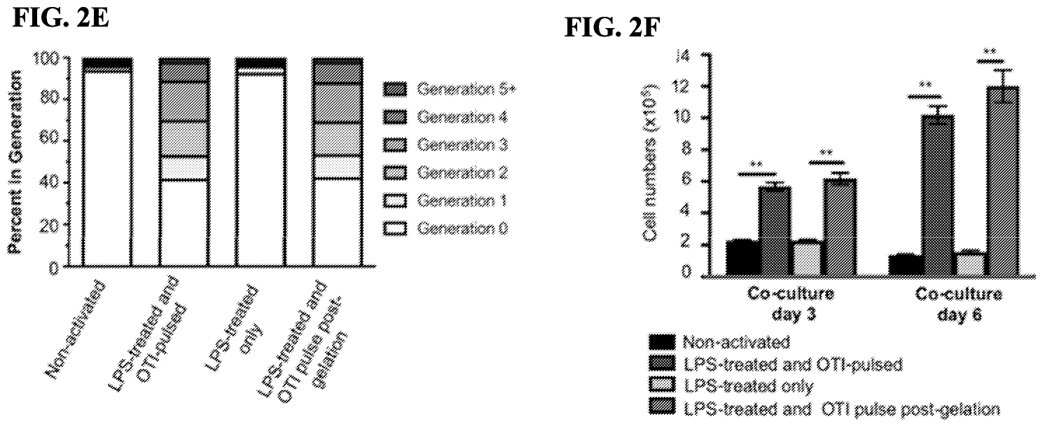

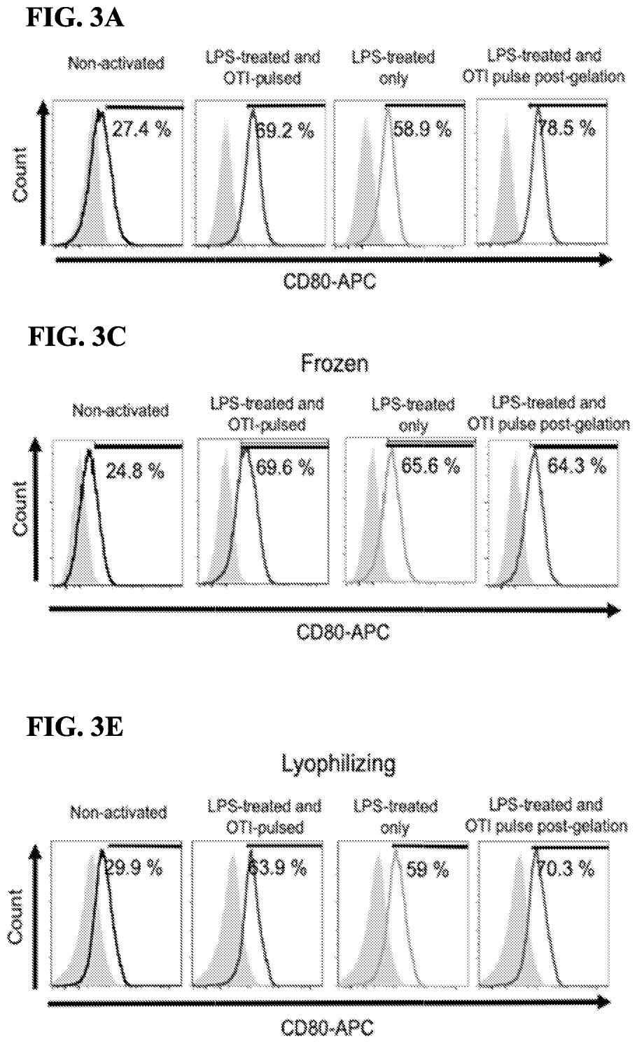

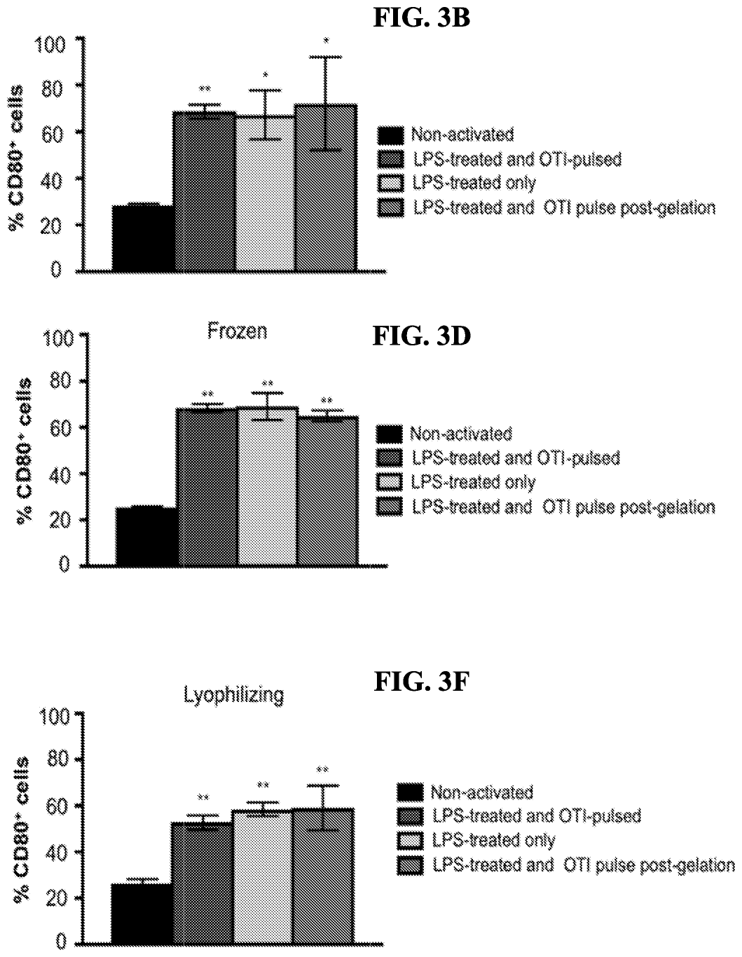

Using flow cytometry, CD80 expression (signal 2) was observed in LPS-treated G-DC, LPS-treated/OTI-pulsed G-DC as well as LPS-treated and OTI pulse post-gelation groups (FIG. 3). This result indicates that both hydrogelation and peptide replacement have little influence on DC surface marker expression. In addition, expression of MHC-I-SIINFEKL complexes (signal 1) were found in 98.1% of LPS-treated/OTI-pulsed G-DCs and in 99.5% of the LPS-treated and OTI pulse post-gelation G-DCs, while only 2.1% of the LPS-treated G-DCs were found to express the MHC-I-SIINFEKL complexes (signal 1) (FIGS. 2B and 2C). These results demonstrate that G-DCs can be successfully loaded with peptide antigen after intracellular gelation and peptide replacement post-hydrogelation did not compromise the membrane-bound lymphocyte activation signals found on G-DCs.

Next, the activity of the peptide antigen-presenting G-DCs on triggering T-cell expansion via the MHC class I-peptide-complex/TCR interaction was investigated by incubating CFSE-labeled CD8⁺ T cells derived from OT-I transgenic mice with G-DCs. Peptide antigen-presenting G-DCs effectively triggered expansion of the target T lymphocytes, showing a similar level of proliferation, generation, and cell number as compared to LPS-treated and OTI-pulsed G-DCs. (FIGS. 2D and 2E). An increased T-cell number was also observed in a time-dependent manner (FIG. 2F), showing that the peptide pulsing post intracellular hydrogelation process does not affect functionality of the G-DCs to modulate antigen-specific T cell response.

Taken together, these data show that antigen presentation can be replaced and modulated on G-DCs post hydrogelation.

(iii) Freezing and Lyophilization Processes Did not Influence the G-DC Functionality

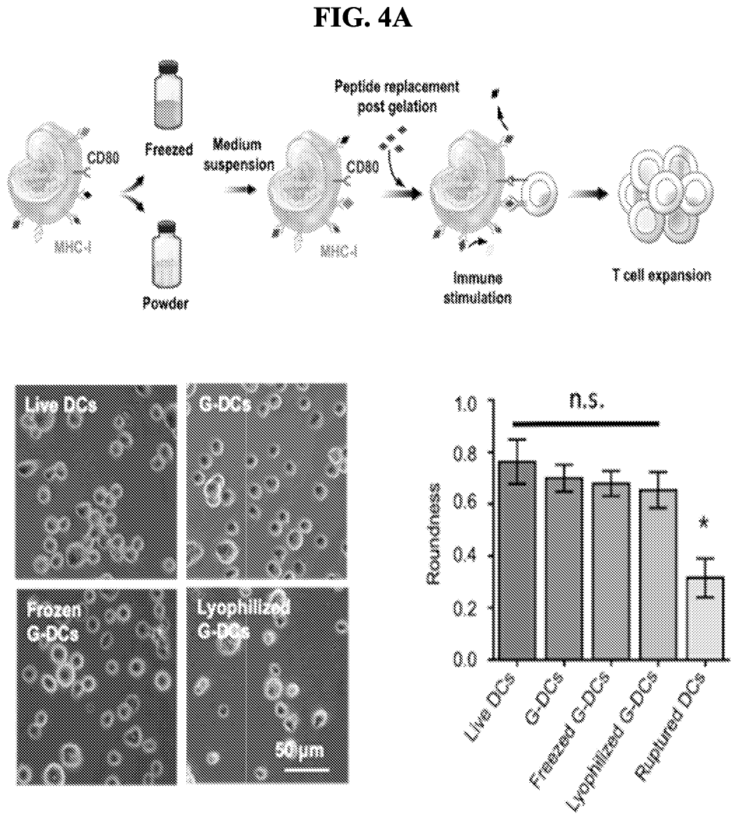

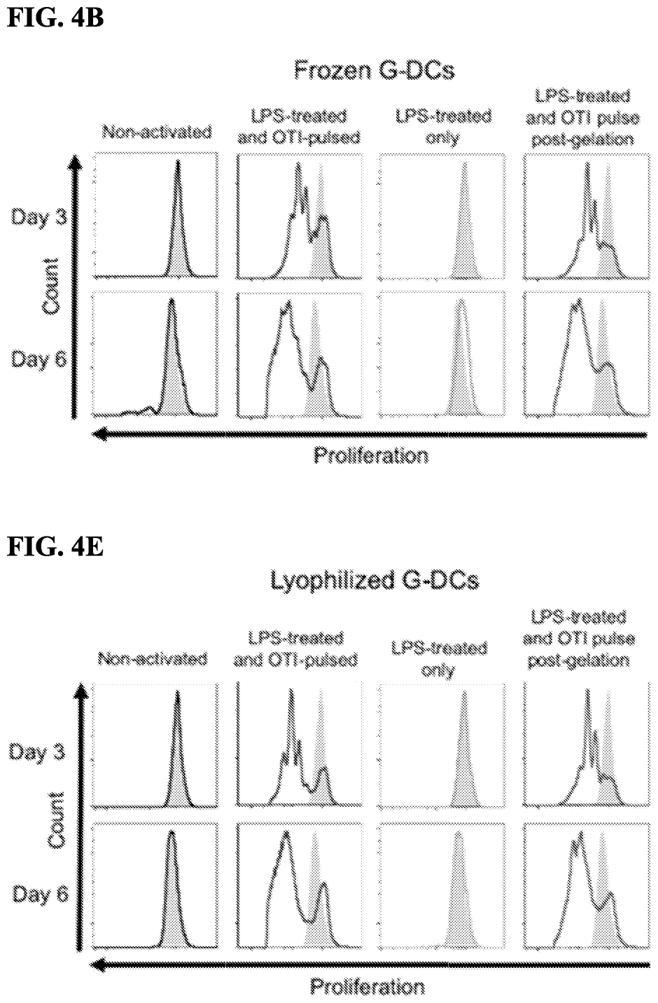

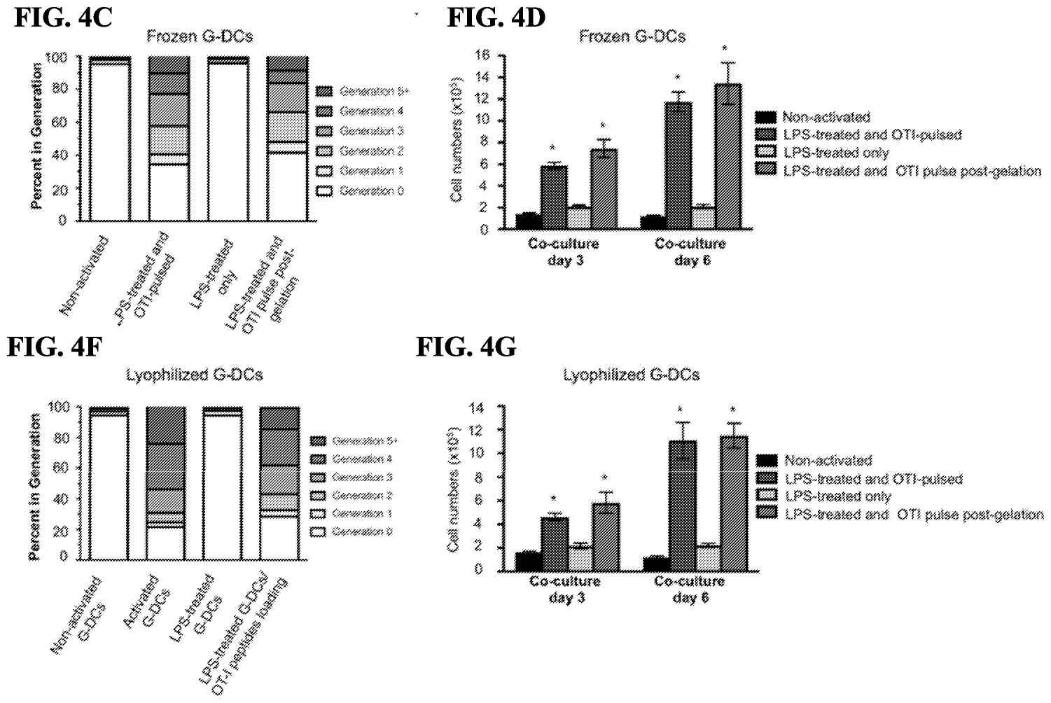

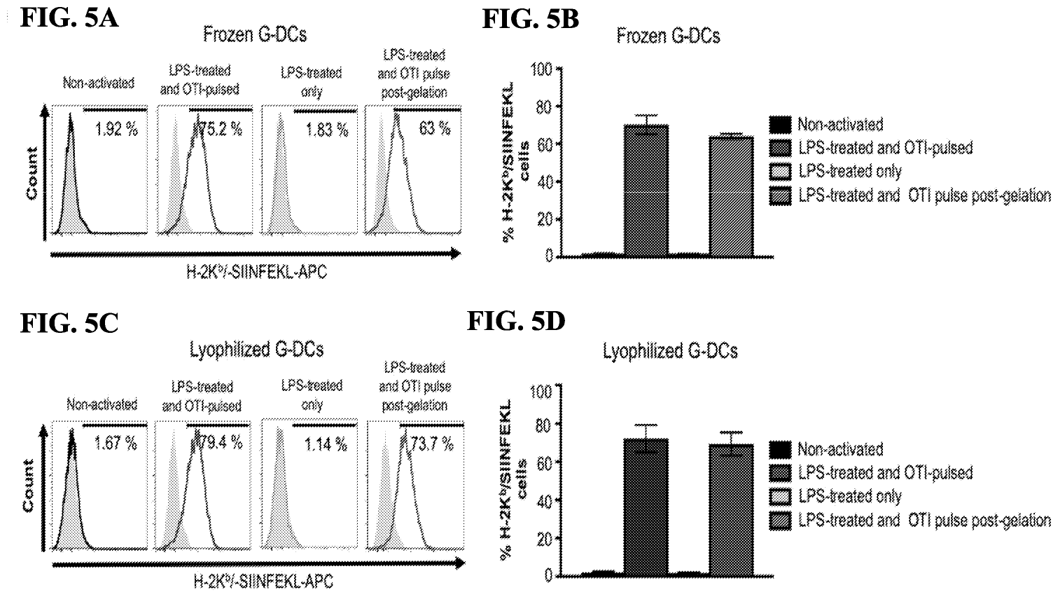

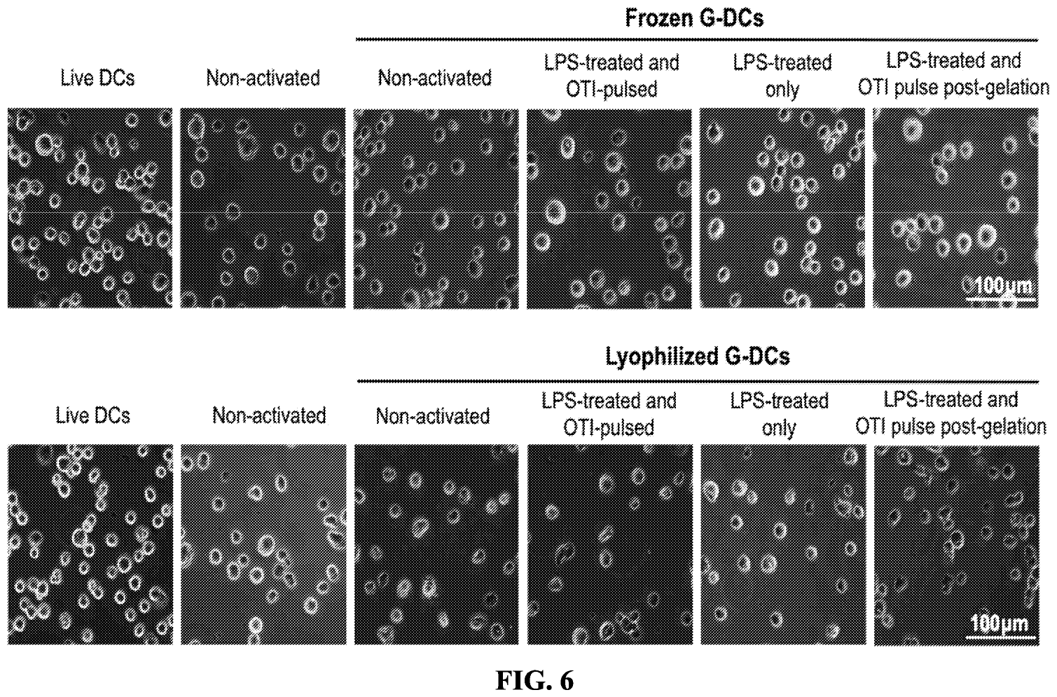

Next, storage of the G-DCs disclosed herein by either freezing or lyophilization was examined. The G-DCs were examined for retention of morphology, surface markers, and functionality after freezing or lymphilization as illustrated in FIG. 4A). The G-DCs of the various groups noted above were first frozen or lyophilized in 10% sucrose and then reconstituted in water. As shown in FIG. 4, G-DCs maintained their shapes after frozen or lyophilization. No structural alternation was observed in control cells, LPS-treated G-DC, LPS-treated/OTI-pulsed G-DC as well as LPS-treated and OTI pulse post-gelation on G-DCs (FIG. 6), indicating cellular morphology of G-DC was effectively preserved after the freezing and/or lyophilizing processes.

The functionality of the frozen and lyophilized samples was further evaluated. To investigate whether the freezing or lyophilizing process had any impact on the biological functions of different G-DCs, the G-DCs of the various groups were examined in an surface marker detection assay (FIGS. 3 and 6) and a T-cell proliferation assay (FIGS. 3B-3G) as disclosed herein. Interestingly, the similar expression pattern of signal 1 (FIG. 6) and signal 2 (FIGS. 3B and 3C) were still maintained in frozen or lyophilized G-DCs, as compared with non-frozen/non-lyophilized G-DCs (FIGS. 3A and 3B). Both frozen and lyophilized G-DCs with LPS-treatment and OTI pulse post-gelation triggered T cell proliferation, resulting in an increased number of OT-I T cells (FIGS. 4B-4G). These results indicate that, after freezing or lyophilization, the G-DCs still retained their bioactivities, for example, activating T cells.

(iv) Gelated Human Dendritic Cells (G-hDCs) Retained MHC-Antigen Complex and Co-Stimulatory Ligand on their Surface Following Storage Upon Freezing or Lyophilizing.

Further, it was demonstrated herein that gelated human dendritic cells prepared by the method disclosed herein showed stability and bioactivity in activating T cells even after freezing or lyophilization.

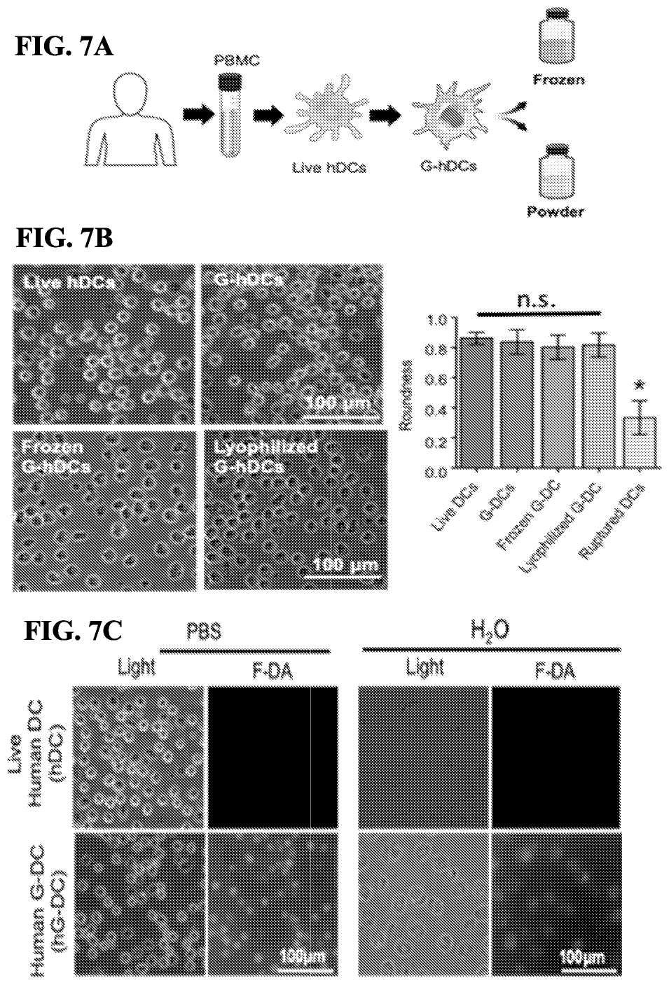

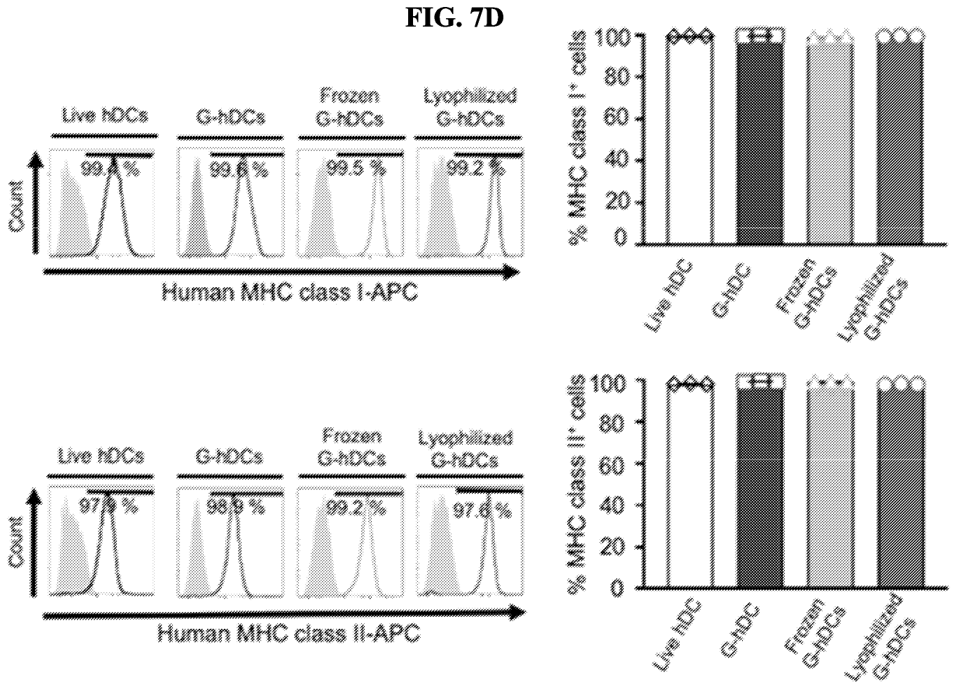

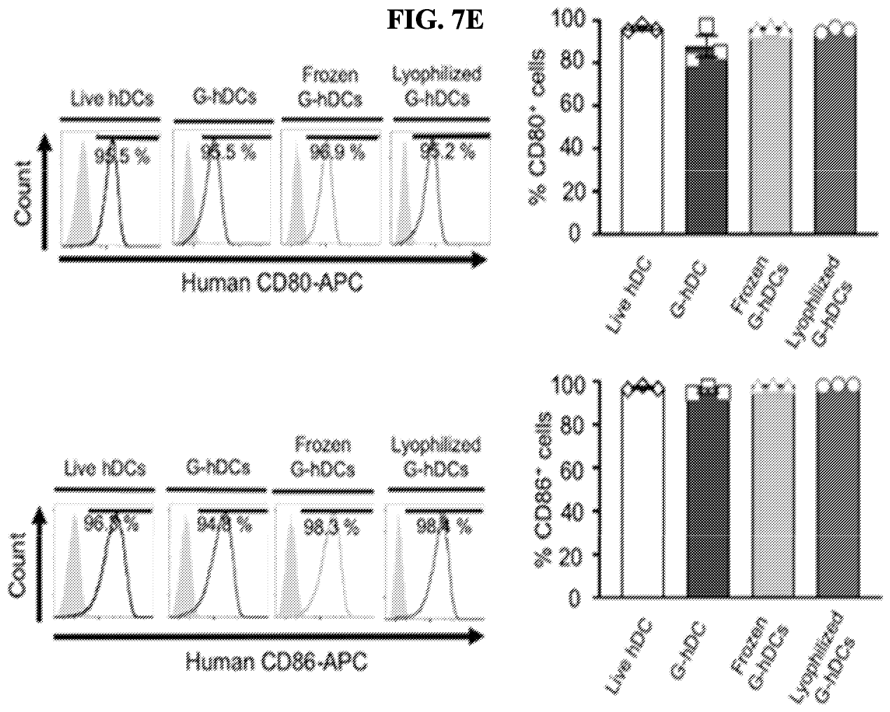

An exemplary experimental procedure is illustrated in FIG. 7A. Bright-field images and fluorescence signals showed the hydrogel network structures within the gelated hDCs at 10% of PEGDA. FIGS. 7B and 7C. This indicates that the hDCs were successfully gelated. Interestingly, due to the successful maintenance of cell membrane integrity on hG-DC, 10 wt % GCs (prepared using 10 wt % PEG-DA) effectively excluded water from entering the cytoplasm to maintain structure (FIG. 7C). To further investigate whether the lymphocyte activation signal 1/2 on hDCs can be properly displayed after gelation of the DCs, human primary dendritic cell was used as the model cells. Using flow cytometry, it was observed that all surface markers, including MHC class I, MHC class II, CD80 and CD80, were expressed on the gelated human DCs at high levels (99.6%, 98.9%, 95.5% and 94.8%, respectively), similar to the expression levels of these surface markers on live human DCs. FIGS. 7D and 7E. Surprisingly, similar expression levels of the surface markers were observed in G-hDCs, frozen G-hDCs, and lyophilized G-hDCs, indicating that freezing and lyophilizing processes did not affect the surface markers on the treated G-hDCs (FIGS. 7D and 7E).

These results demonstrate the feasibility of preparing gelated human primary DCs as artificial antigen presenting cells (aAPCs), which may be used for clinical applications.

### Example 2: Cytokine-Release System Comprising Gelated Dendritic Cells and Microparticles

This example describes a cytokine-release system comprising the gelated dendritic cells (G-DCs) prepared as described in Example 1 above associated with microparticles encapsulating cytokines.

### Methods

(i) Synthesis of PLGA Micro Particles

PLGA microparticles (MPs) containing dye were synthesized by double-emulsion method followed by solvent evaporation. PLGA with different viscosities were used, here we denote the PLGA with inherent viscosity 0.15-0.25 dL/g as PLGA(A), and that with viscosity 0.55-0.75 dL/g as PLGA(B). A series of polymer weight ratio was carried out:
PLGA(A):PLGA(B)=100:0, 65:35, 50:50, 35:65, or 20:80.

Briefly, PLGA was dissolved in dichloromethane (DCM) (100 mg/ml) to form to form oil phase and red dye was dissolved in 100 mM pH8 Na₂HPO4 to from aqueous phase. 250 μL of the oil phase and 10 μL of the water phase (volume ratio=25:1) were then emulsified by a probe sonicator in ice under the pulse mode with 40% amplitude and on-off durations of 1 and 2 s for 1 min. The emulsion was then added dropwise to a fast-stirring buffer (10 mM pH8 Na₂HPO4, stirred at 1000 rpm) of 5 mL in volume in an ice bath to disperse the micro-droplets while preventing DCM evaporation. The emulsion was stirred at 1000 rpm for 5 minutes. After that, the emulsion was heated at 35° C. for 35 minutes to evaporate DCM and the stirring rate during the heating process was adjusted depending on the polymer ratio. For PLGA(B)≥50% (PLGA(A):PLGA(B)=50:50, 35:65, or 20:80), stirring rate was kept at 1000 rpm. For polymer B<50% (PLGA(A):PLGA(B)=100:0, or 65:35), the stirring rate was lower down to 500 rpm. Following that, the stirring rate in all groups were lower down to 200 rpm was kept for 20 minutes to prevent the destruction of particles during DCM evaporation. After DCM was fully evaporated, the microparticles were washed twice by centrifugation at 500 rpm for 3 min followed by resuspension of the pellet in 1 mL of pH 7 PBS. Finally, the particles were preserved in pH7 phosphate buffer.

(ii) Encapsulation Efficiency Determination

Encapsulation efficiency (EE) was calculated using the formula below:

EE=dye (mg) in particle/total dye (mg).

The particles were broken down by 80% acetone followed by solvent evaporation. After the solvent was evaporated, water was added to dissolve the dye. The broken pieces of PLGA were removed by centrifugation at 30,000 g. The supernatant was collected and detected by light spectrum (512 nm) to evaluate the dye concentration and the amount of dye in particles was therefore obtained.

(iii) Release Study

The release of dye was studied at 37° C. and 4° C. In order to study the release in human body, 3 mg of MPs were suspended in 10 ml pH7.4 PBS at 37° C. in water bath at the stirring rate of 150 rpm. To determine the amount of released dye, the MPs were settled down for 10 mins and the PBS supernatant was collected at certain periods of time. The PBS was analyzed by light spectrum at the wavelength of 512 nm. The 4° C. release study was conducted by keeping 2 mg particles in 2 ml pH7 phosphate buffer. Likewise, the PBS supernatant was collected for light-absorption analysis. The buffer in both studies were renewed within 12 hours to ensure the consistency of pH value.

### Results

(i) GCs-MPs Complex Served as Cytokine-Releasing Gelated Cellular Spheroids

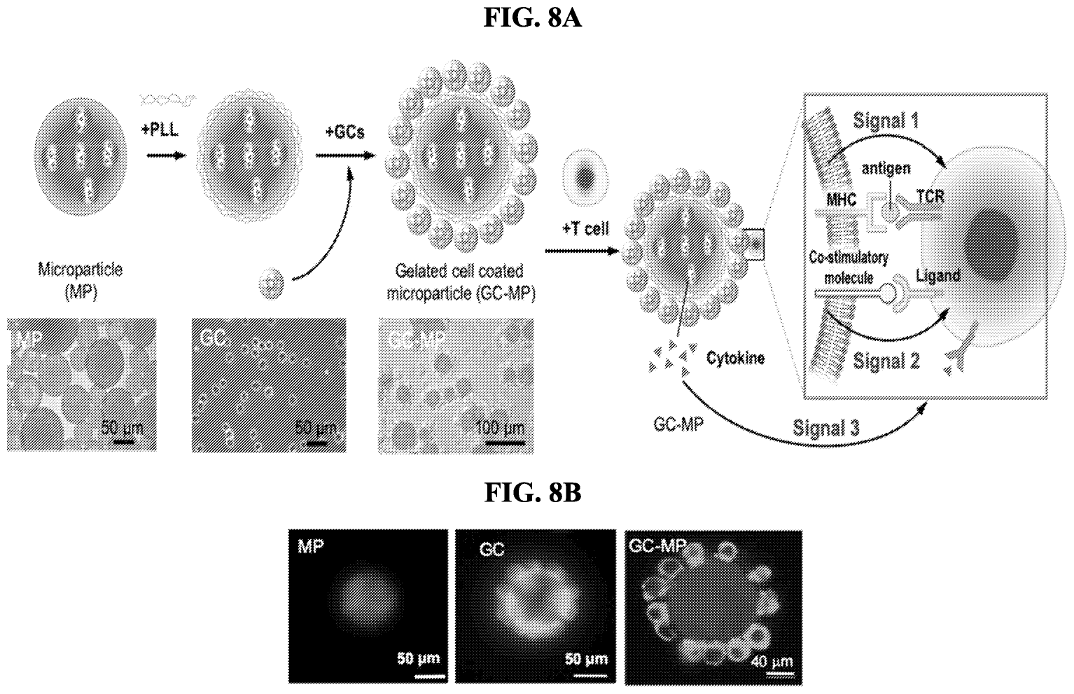

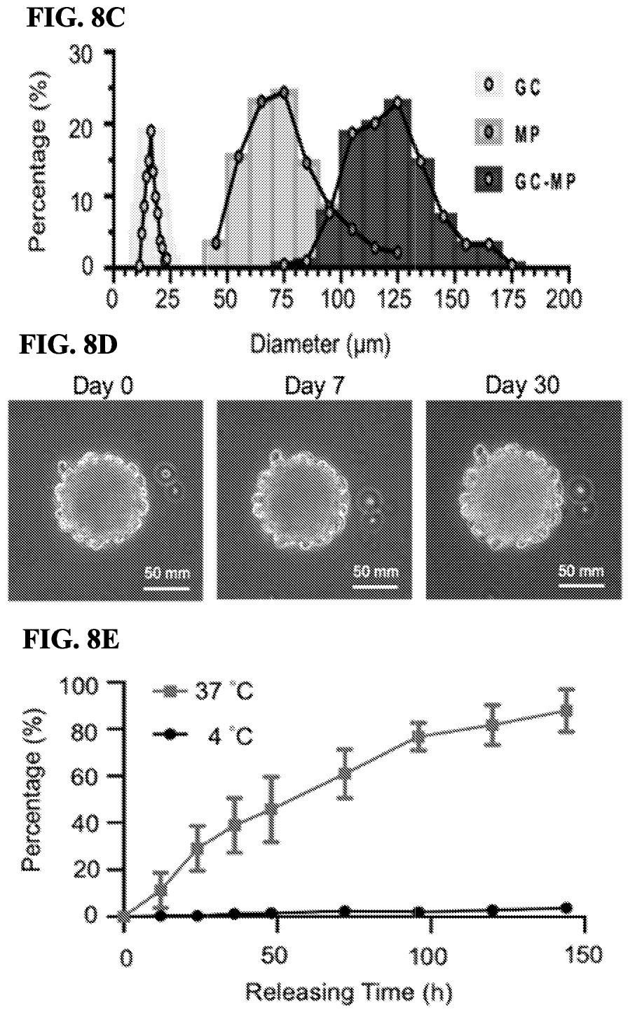

Live antigen presenting cells (APCs) secrete lymphocyte-activating cytokines, which plays a major role in T cell activation. Reported herein is a cytokine-release system (GC-MPs) comprising gelated dendritic cells complexed with microparticles encapsulating cytokines (using IL2 as an example) to mimic live APCs. As illustrated in FIG. 8A, GC-MPs composed of PLGA-based MPs and G-DCs would possess components for triggering all three lymphocyte activation signals for T cell activation, i.e., primary signaling, secondary signaling, and cytokine signaling.

MPs were prepared by a double emulsion process disclosed above. A hydrophilic sulfo-Cy5 dye was used as an exemplary cargo to validate successful cargo encapsulation and release. As shown in FIGS. 8A and 8B, cargo loading into the MPs were observed by both bright-field (FIG. 4A) and fluorescence microscopy. Complexing G-DCs and the cargo-loaded MPs was mediated using poly-lysine. Successful complexing was observed using confocal microscopy. FIG. 8A. The size distribution of G-DCs, MPs, and GC-MPs were 20, 75 and 125 um, respectively (FIG. 8C). Notably, the G-DCs remained firmly attached to the surface of MPs during storage for 30 days (FIG. 8D). In vitro release profiles of GC-MPs, the IL-2 slowly released at 37° C. but not at 4° C.

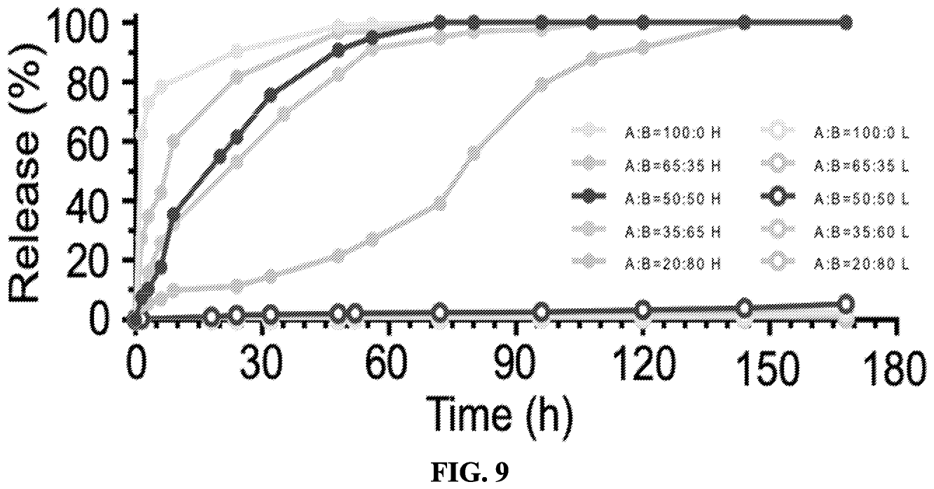

The IL-2 release kinetics are provided in FIG. 9. As shown below, release of IL-2 from the GC-MPs contributed to fully activation of T cells.

Taken together, the results showed successful construction of an artificial APC system comprising all components needed for triggering the three activation signaling pathways for fully activation of T cells. Such an artificial APC system also has controlled release capacity for cytokine secretion.

(ii) GC-MPs Acted as aAPC-Mimetic Biomaterials that Successfully Induced CD8 T Cell Activation and Proliferation

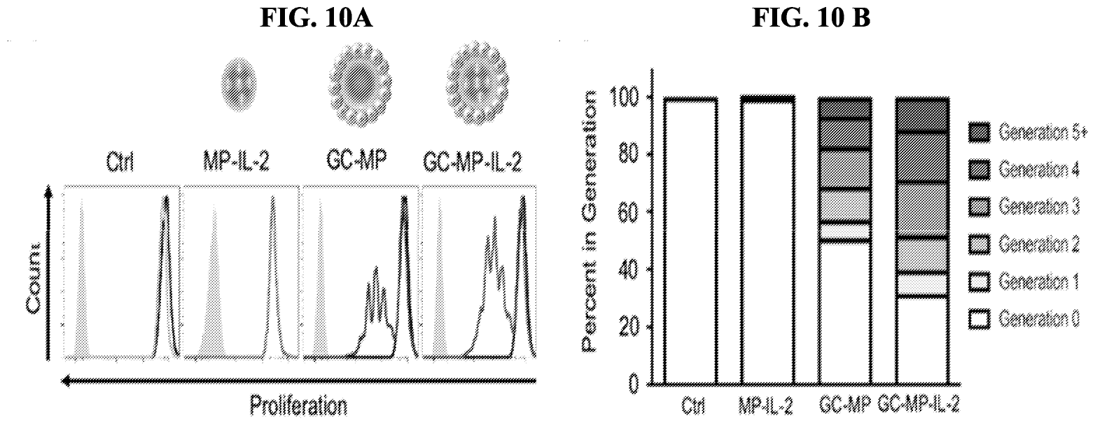

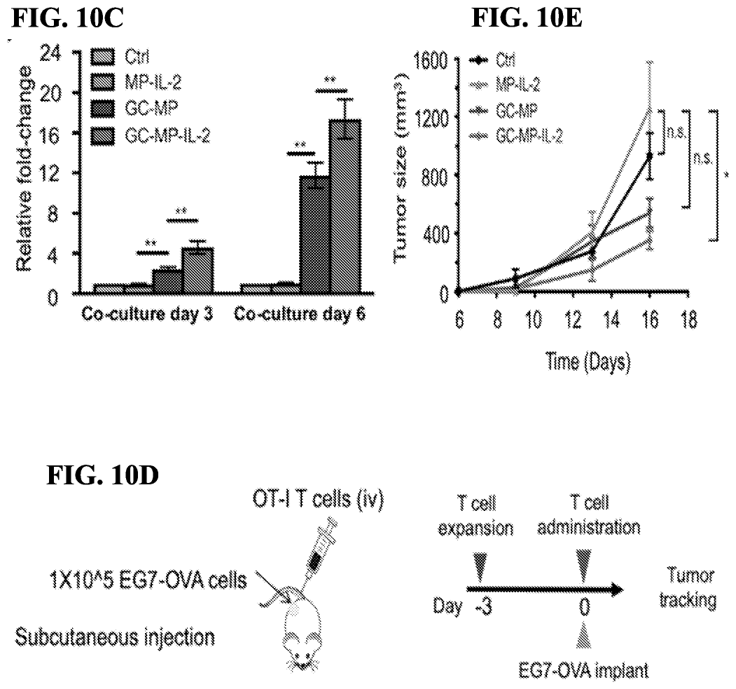

The activity of the GC-MP system disclosed herein for activating CD8+ T cells was examined. First, whether the GC-MPs could fully activate CD8 T cells was investigated. After CFSE-labeled OT-I T cells were co-cultured with MP-IL-2, GC-MPs or GC-MP-IL-2, CD8 T cell proliferation was detected by flow cytometry. An increase in the proliferation and number of T cells in the GC-MP and GC-MP-IL-2 groups, but not in the MP-IL-2 group, was observed. FIGS. 10A-10C. This indicates that the GC-MP structure did not affect the immune-regulation function of the G=DCs. Notably, GC-MP-IL-2 (triggering all three signaling pathways) resulted in higher T cell expansion as compared to the GC-MP (triggering only the primary and co-stimulatory signaling pathways). FIGS. 10A-10C. This difference indicates that the cytokine-release function of the GC-MPs is important in activating immune cells. Thus, the G-DCs disclosed herein can be used in combination with other suitable platforms to regulate immune responses.

To further investigate the potency of GC-MPs in improving adoptive T cell therapy, GC-MP-expanded OTI-specific CD8 T cells were further assessed in vivo in mice transplanted with EG7-OVA (FIG. 10D). The results showed that the CD8 T cells activated by the GC-MP-IL-2 aAPC system significantly reduced tumor size in vivo, compared to other groups. FIG. 10E.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. An artificial antigen presenting cell complex comprising:
(a) one or more gelated human dendritic cells; and
(b) a controlled release system associated with one or more cytokines;
wherein the one or more gelated human dendritic cells are attached to the controlled release system.

2. The artificial antigen presenting cell complex of claim 1, further comprising an antigenic peptide displayed on the surface of the one or more gelated human dendritic cells.

3. The artificial antigen presenting cell complex of claim 1, wherein the controlled release system comprises a microparticle, which encapsulates the one or more cytokines.

4. The artificial antigen presenting cell of claim 1, wherein the one or more cytokines comprise IL-2, IL-3, IL-4, IL-6, IL-7, IL-9, IL-12, IL-15, IL21, IL-23, IL-10, TNF-α, IFN-α, IFN-β, IFN-γ, IFNλ, granulocyte-macrophage colony-stimulating factor, or a combination thereof.

5. The artificial antigen presenting cell complex of claim 1, wherein the microparticle comprises one or more degradable polymers.

6. The artificial antigen presenting cell complex of claim 5, wherein the one or more degradable polymers comprise poly(lactic-co-glycolic acid), polylactic acid, polycaprolactone, polyurethane, polyacrylate, or a combination thereof.

7. The artificial antigen presenting cell complex of claim 2, wherein the antigenic peptide is displayed by a major histocompatibility complex class I molecule on the gelated human dendritic cell.

8. The artificial antigen presenting cell complex of claim 1, wherein the gelated human cells comprise intracellular cross-linked polyethylene glycol diacrylate (PEG-DA).

9. A method for producing a gelated human dendritic cell, the method comprising:
(a) providing a population of human dendritic cells;
(b) permeating a gelation buffer into the human dendritic cells, wherein the gelation buffer comprises a photo initiator, a polymer or a polymerizable monomer, and dimethyl sulfoxide; and
(c) exposing the human dendritic cells permeated with the gelation buffer to a light to cause crosslinking the polymer, thereby forming the gelated human dendritic cells.

10. The method of claim 9, further comprising:
(d) coupling the gelated human dendritic cells to a controlled release system, which is associated with one or more cytokines.

11. The method of claim 9, wherein the gelation buffer comprises the polymer, which is an acrylic polymer.

12. The method of claim 11, wherein the acrylic polymer comprises poly(ethylene glycol) diacrylate (PEG-DA).

13. The method of claim 12, wherein the acrylic polymer is PEG-DA having an average molecular weight of about 250 to about 2,000 dalton.

14. The method of claim 13, wherein the PEG-DA has an average molecular weight of about 600 to about 800 dalton.

15. The method of claim 9, wherein the gelation buffer comprises the polymerizable monomer.

16. The method of claim 15, wherein the polymerizable monomer is 2-hydroxyethyl methacrylate, (hydroxyethyl)methacrylate, acrylic acid, sodium acrylate, ethylene glycol dimethacrylate, or a combination thereof.

17. The method of claim 9, wherein the photo initiator is 2-hydroxy-4′-(2-hydroxyethoxy)-2-methylpropiophenone.

18. An artificial antigen presenting human dendritic cell, which is produced by a method of claim 9.

19. A method of activating an immune cell, comprising:
(i) providing an artificial antigen presenting cell complex of claim 18;
(ii) priming the artificial antigen presenting cell complex with an antigenic peptide to display the peptide on the surface of the gelated human dendritic cell in the artificial antigen presenting cell complex; and
(iii) contacting the artificial antigen presenting cell complex produced in step (with a cell population comprising immune cells to activate the immune cells.

20. The method of claim 19, wherein the immune cells comprise T cells.

21. The method of claim 17, wherein the peptide is derived from a viral antigen, a bacterial antigen, a fungal antigen, a parasite antigen, a cancer antigen, or a self antigens associated with an autoimmune disease.

22. The method of claim 19, further comprising administering the activated immune cells produced in step (iii) to a subject in need thereof.
